(19) United States
(12) Patent Application Publication
(10) Pub. No.: US 2025/0065057 A1
(43) Pub. Date: Feb. 27, 2025

(54) A sub-assembly of a medicament cassette unit

(71) Applicant: SHL Medical AG, Zug (CH)

(72) Inventors: Daniel Carlsson, Nacka Strand (SE); Chi-Chia Tung, Taoyuan City (TW); Ming-Ting Yin, Taoyuan City (TW); Johan Zander, Nacka Strand (SE); Oscar Alexandersson, Nacka Strand (SE)

(21) Appl. No.: 18/724,354

(22) PCT Filed: Dec. 20, 2022
(86) PCT No.: PCT/EP2022/086956

§ 371 Date: Jun. 26, 2024

Related U.S. Application Data
(60) Provisional application No. 63/419,561, filed on Oct. 26, 2022.

(30) Foreign Application Priority Data
Jan. 3, 2022 (EP) 22150025.9

(51) Int. Cl. A61M 5/32

(52) U.S. Cl. CPC A61M 5/3245; A61M 5/3202

(57) ABSTRACT

The present disclosure provides a sub-assembly of a medicament cassette unit, the sub-assembly including a tubular housing, a delivery member cover, and a cap. The tubular housing extends along a longitudinal axis between a proximal end and a distal end. The tubular housing is configured to receive a medicament container. The delivery member cover comprises a tubular section arranged telescopically relative to the proximal end of the tubular housing. The delivery member cover comprises a distally directed surface. The cap is releasably attached to the tubular section of the delivery member cover, and the cap comprises a proximally directed surface adjacent to the distally directed surface of the delivery member cover.

### CROSS-REFERENCE TO RELATED APPLICATIONS

The present application is a U.S. National Phase Application pursuant to 35 U.S.C. § 371 of International Application No. PCT/EP2022/086956 filed Dec. 20, 2022, which claims priority to (i) European Patent Application No. 22150025.9 filed Jan. 3, 2022, and (ii) U.S. Provisional Patent Application No. 63/419,561 filed Oct. 26, 2022. The entire disclosure contents of these applications are herewith incorporated by reference into the present application.

### TECHNICAL FIELD

The present disclosure generally relates to a sub-assembly of a medicament cassette unit, and particularly to a sub-assembly comprises a housing, a delivery member cover, and a cap.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone.

It is an advantage when the medicament delivery devices for the self-administration comprise automatic functions so that even if users are without professional training or are in an emergency situation, the users can easily and properly use the medicament delivery devices.

Even though many of the devices on the market, as well as the ones described above, have their respective advantages, there is still room for improvements.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament cassette unit, the sub-assembly comprising: a tubular housing, a delivery member cover, and a cap; the tubular housing extends along a longitudinal axis between a proximal end and a distal end; the tubular housing is configured to receive a medicament container; the delivery member cover comprises a tubular section arranged telescopically relative to the proximal end of the tubular housing; the delivery member cover comprises a distally directed surface; the cap is releasably attached to the tubular section of the delivery member cover; the cap comprises a proximally directed surface adjacent to the distally directed surface of the delivery member cover; at least one of the distally directed surface and the proximally directed surface is a chamfer surface bevelled relative to the longitudinal axis; the distally directed surface is formed on a protrusion that extends from a flexible wall of the delivery member cover in a direction transverse to the longitudinal axis or the proximally directed surface is formed on a protrusion that extends from a flexible wall of the cap in the direction transverse to the longitudinal axis; and the delivery member cover is axially movable relative to the tubular housing from a distal position where a support surface of the tubular housing is aligned with an outer surface of the flexible wall in the direction transverse to the longitudinal axis and a proximal position where the support surface of the tubular housing is closer to the distal end of the housing than the flexible wall.

Preferably, according to another embodiment, the flexible wall is defined by a flexible arm extending in the direction of the longitudinal axis.

Preferably, according to another embodiment, the cap comprises the flexible arm extending towards the distal end of the housing.

Preferably, according to another embodiment, the distally directed surface of the delivery member cover extends from an outer surface of the tubular section of the delivery member cover.

Preferably, according to another embodiment, the sub-assembly comprising a biasing member extending along the longitudinal axis between a proximal end and a distal end; and the distal end of the biasing member is adjacent to a proximally directed surface of the tubular housing and the proximal end of the biasing member is adjacent to a distally directed surface of the delivery member cover.

Preferably, according to another embodiment, the delivery member cover comprises a ledge extending in the direction transverse to the longitudinal axis; and wherein the distally directed surface of the delivery member cover is defined by the ledge.

Preferably, according to another embodiment, the cap comprises a gripping arm extending towards the distal end of the tubular housing; the gripping arm comprises a proximally directed surface configured to engage with a delivery member sheath of a medicament container.

Preferably, according to another embodiment, the gripping arm of the cap is arranged within the delivery member cover; and the flexible wall of the cap is arranged between the tubular housing and the delivery member cover.

Preferably, according to another embodiment, the delivery member cover comprises a flexible section at a distal end of the delivery member cover; and wherein the flexible section comprises a protrusion extending from the delivery member cover towards the tubular housing in the direction transverse to the longitudinal axis.

Preferably, according to another embodiment, a cut-out or recess is arranged in a wall of the tubular housing; and the flexible section is movable in the direction transverse to the longitudinal axis relative to the delivery member cover between an engaged position where the protrusion of the flexible section is positioned within the cut-out or recess of the tubular housing and a released position where the protrusion of the flexible section is positioned out of the cut-out or recess of the tubular housing.

Preferably, according to another embodiment, the flexible section of the delivery member cover is moved from the engaged position where the protrusion of the flexible section is positioned within the cut-out to the released position where the protrusion of the flexible section is positioned out of the cut-out when the protrusion of the flexible section is pressed from the outside of the tubular housing.

Preferably, according to another embodiment, the flexible section of the delivery member cover is configured to be moved from the engaged position to the released position by a section of a drive unit of the medicament delivery device when the cassette unit with the sub-assembly is attached to the drive unit.

Preferably, according to another embodiment, the flexible section is moved by the drive unit via the protrusion.

Preferably, according to another embodiment, the protrusion is configured to extend out from the housing through the cut-out of the housing.

In another aspect of the invention, a method of operating a medicament delivery device is provided. The method comprises the following steps in the following order: providing a medicament cassette unit of the medicament delivery device, wherein the medicament cassette unit comprises the sub-assembly of the invention; providing a drive unit of the medicament delivery device, wherein the drive unit comprises a section for receiving a part of the medicament cassette unit; inserting the part of the medicament cassette unit into the section of the drive unit, thereby engaging the flexible section of the delivery member cover of the medicament cassette unit with an inner surface of the drive unit; moving the delivery member cover from the distal position to the proximal position; and pulling the cap in the proximal direction relative to the tubular housing, thereby removing the cap from the tubular housing.

Preferably, according to another embodiment, the sub-assembly is used in a medicament delivery device comprising a multiple-chamber medicament container arranged within the carrier.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery member is an injection needle or a spray nozzle.

Preferably, according to another embodiment, the medicament container is a syringe or a cartridge.

Generally, the medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the medicament) and a pharmaceutically acceptable carrier. Pharmaceutical formulations comprising a drug as listed herein (or a pharmaceutically acceptable salt of the medicament) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament cassette unit comprising a sub-assembly of the invention.

FIG. 2 schematically shows a medicament delivery device formed by a drive unit and the medicament cassette unit of FIG. 1.

FIG. 3 schematically shows a perspective view of components of the sub-assembly of FIG. 1.

FIG. 4 schematically shows a perspective view of a housing and a delivery member cover of the sub-assembly of FIG. 1.

FIG. 5 schematically shows a perspective view of a rear end of the housing and the delivery member cover of FIG. 4.

FIG. 6 schematically shows a perspective view of a cap of the sub-assembly of the invention of FIG. 1.

FIG. 7 schematically shows a perspective view of the cap of FIG. 6 and a proximal end of the delivery member cover of FIG. 4.

FIG. 8 schematically shows a perspective view of the housing of FIG. 4.

FIGS. 9-10 schematically show cross-section views of a proximal end of the sub-assembly.

FIGS. 11-12 schematically show perspective views of a lockout structure between the delivery member cover of the sub-assembly of FIG. 1 and the housing of the sub-assembly of FIG. 1.

FIGS. 13-16 schematically show perspective views of a proximal end of the sub-assembly in another example.

FIG. 17 schematically shows a perspective view of a lock ring of the sub-assembly in another example and the delivery member cover of the sub-assembly in another example.

### DETAILED DESCRIPTION

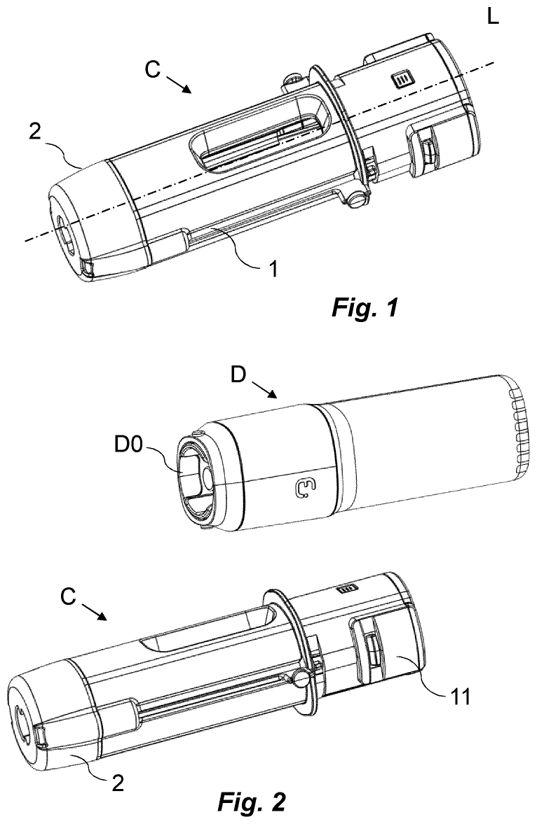

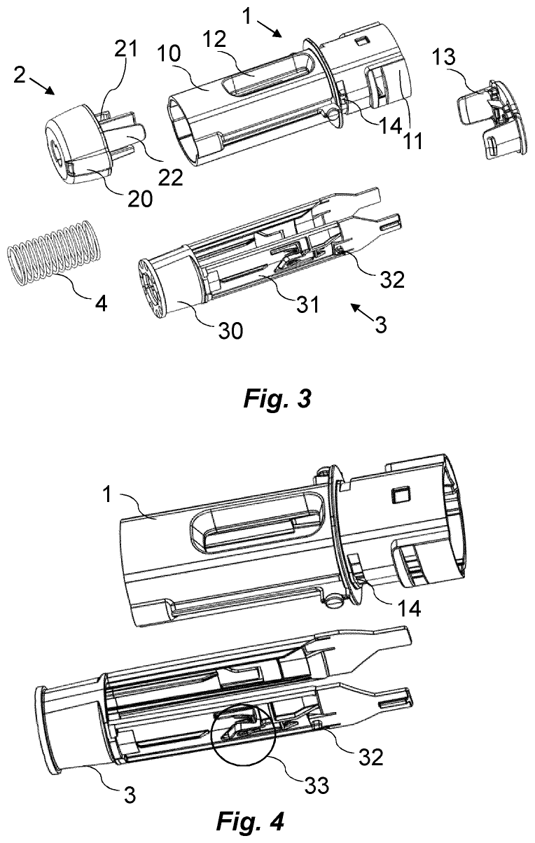

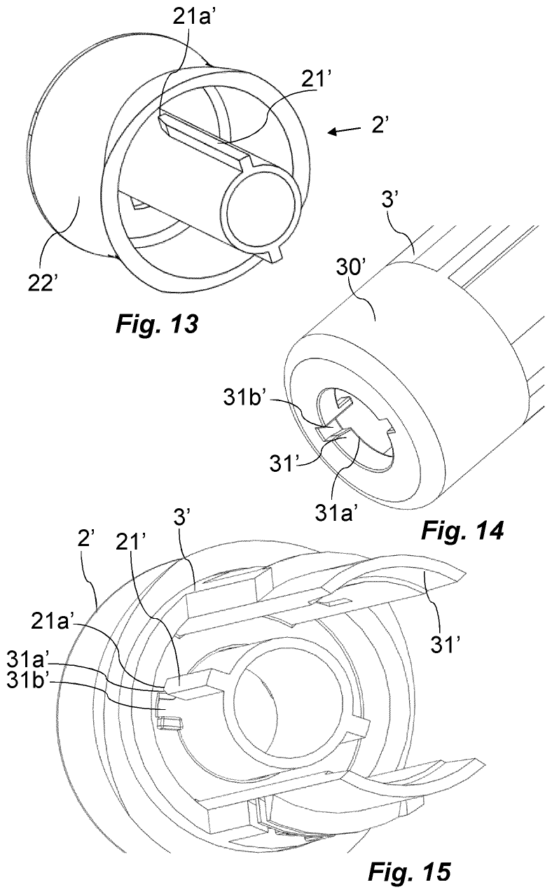

FIGS. 1-17 illustrate a sub-assembly of a medicament cassette unit. The term “medicament delivery device cassette unit” will sometimes be abbreviated as “cassette unit” in this description. It should be noted that both the term “medicament delivery device cassette unit” and the term “cassette unit” are used with the same meaning in this description.

The cassette unit is usually used with a medicament delivery device with two detachable parts, as shown in FIG. 2. There can be certain technical demands for making a medicament delivery device, such as a controllable medicament delivery speed; a high output force to deliver a high volume and/or high viscosity medicament; multiple indications for different medicament delivery stages; and/or data recording and/or communication, for example, to track prescription adherence. However, the cost of manufacturing medicament delivery devices that can perform all functions and fulfil the technical demands described above is typically high, which makes the use of such medicament delivery devices for frequent treatments less affordable. Medicament delivery devices with two detachable parts are usually made with a reusable part and a disposable part. The reusable part comprises most of the mechanical and/or electronic components. For example, the reusable part normally comprises a powerpack, namely, an assembly that comprises a power source and components that are configured to transfer the output force from the power source to a medicament. The disposable part is usually without electronics to lower the cost of manufacture. The disposable part, commonly referred to as a cassette unit C, is usually designed to accommodate a medicament container, e.g. a syringe or cartridge, that has a medicament contained within. The syringe or cartridge can be made of glass or plastic. The syringe or cartridge may comprise a rubber stopper sealing a distal end of the syringe or cartridge, and a septum or medicament delivery member with a rubber sheath sealing a proximal end of the syringe or cartridge. Alternatively, the syringe or cartridge may be collapsible with only a septum or medicament delivery member (i.e. no stopper) with a rubber delivery member sheath sealing a proximal end of the syringe or cartridge. It should be noted that the septum or the rubber sheath usually provides a sterile seal for the medicament container of the cassette unit. The sterile seal is configured to be broken right when a user wants to carry out a medicament delivery operation.

The reusable part of the medicament delivery device, (i.e., a drive unit D), usually comprises a driver for expelling the medicament contained within the medicament container in the proximal direction of the medicament delivery device. The drive unit D may comprise a plunger rod for pushing on the medicament container. In one example, the stopper of the medicament container can therefore be moved in the proximal direction relative to the rest of the medicament container. In another example, the plunger rod may collapse the medicament container by pushing the medicament container in the proximal direction relative to the cassette unit C. The drive unit D also comprises a power source, such as a spring, a gas canister, or a motor-driven gear set, for providing the power required to expel the medicament. The focus of the concept described herein is a sub-assembly for the medicament delivery device cassette, not the drive unit, and various types of drive units could be used with the cassette units with the sub-assembly described herein. As such, the drive unit will not be comprehensively described.

The sub-assembly of the invention provides a safety mechanism configured to prevent the user from breaking the sterile seal of the medicament container of the cassette unit before attaching the cassette unit to the drive unit. The sub-assembly also provides a mechanism that a delivery member cover is moved in a proximal direction relative to the tubular body when the cassette unit is attached to the drive unit D (the cassette unit comprises the sub-assembly of the invention). The proximal movement of the delivery member cover is configured to unlock the safety mechanism.

The sub-assembly comprises a tubular housing 1; 1′, a cap 2; 2′, and a delivery member cover 3; 3′. The tubular housing 1; 1′ extends along a longitudinal axis L between a proximal end and a distal end.

The tubular housing 1; 1′ is configured to receive the medicament container of the cassette unit. The tubular housing 1; 1′ comprises a housing body 10; 10′ and a distal lid 13. In a preferred example, the medicament container is configured to be inserted into the housing body 10 through the distal end of the housing body 10. The step of inserting the medicament container into the housing body 10 can be carried out during the assembly step in a medicament delivery device manufacture site, or by a caregiver, e.g., a pharmacist or a nurse. After the medicament container is inserted into the housing body 10, the distal lid 13 is configured to attach to the distal end of the housing body 10, so that the medicament container will not fall from the distal end of the housing 1. An aperture is arranged in the distal lid 13, so that a plunger rod from the drive unit can move into the housing 1 for expelling the medicament within the medicament container.

In a preferred example, the tubular housing 1 comprises a fastener 11 configured to engage with a counter fastener DO of the drive unit D, so that the cassette unit is attached to the drive unit. In a preferred example, the fastener 11 is on the distal end of the housing 1. The fastener 11 and the counter fastener DO can form a bayonet engagement, a screw thread engagement, or a snap-fit engagement, dependent on the design of the medicament delivery device, as shown in FIG. 2. Alternatively, the sub-assembly comprises a lock ring 5 attached to the housing 1′. The lock ring 5 is axially immovable and rotatable around the longitudinal axis L relative to the housing 1′. In this example, the lock ring 5 comprises a fastener 51 configured to be engaged with the counter fastener DO of the drive unit, as shown in FIG. 17. In a preferred example, the fastener 51 and the counter fastener DO formed a bayonet connection.

In a preferred example, a window 12 is arranged in the wall of the housing body 10, so that the user can observe the medicament through the window 12.

The delivery member cover 3; 3′ comprises a tubular section 30; 30′ is arranged telescopically relative to the proximal end of the housing 1; 1′. The tubular section 30; 30′ of the delivery member cover 3; 3′ is configured to cover the medicament delivery member of the cassette unit, e.g., an injection needle. In a preferred example, the delivery member cover 3; 3′ comprises an arm 31; 31′ extending from the tubular section 30; 30′ towards the distal end of the housing 1; 1′.

In a preferred example, the delivery member cover 3 comprises a flexible section 32 at a distal end of the delivery member cover 3. Preferably, the flexible section is a part of the arm or is extending from the arm 31. The flexible section comprises a protrusion 32 extending from the delivery member cover 3 towards the tubular housing 1 in the direction transverse to the longitudinal axis L, as shown in FIG. 4.

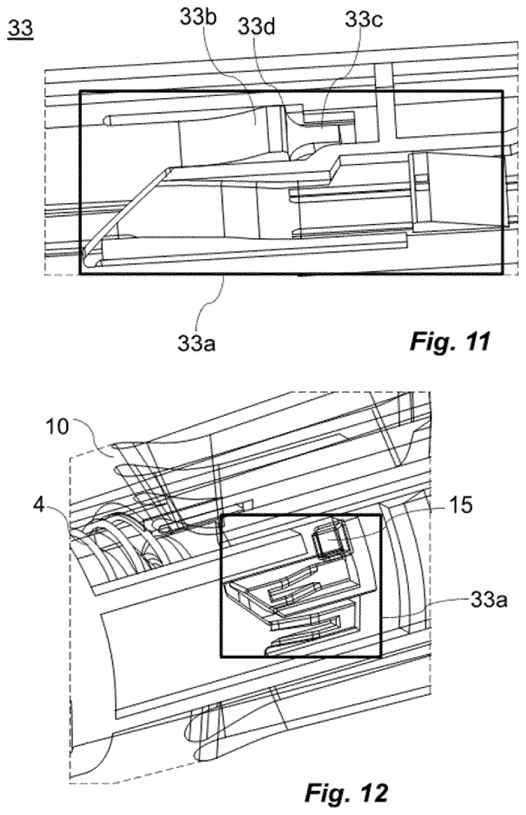

Furthermore, in a preferred example, the delivery member cover 3 comprises a lockout structure 33, as shown in FIG. 4 and FIG. 11. The delivery member cover 3 is configured to be moved in the distal direction by the user such that the medicament delivery member of the cassette unit is exposed and thus the medicament delivery operation can be carried out. When the medicament delivery operation is completed, the user can lift the delivery member cover 3 from a medicament delivery site, the delivery member cover 3 can move in the proximal direction relative to the housing 1 to cover the medicament delivery member again. The lockout structure 33 is configured to block the delivery member cover 3 from moving in the distal direction relative to the housing 1 after the user lift the delivery member cover 3 from the medicament delivery site so that the used medicament delivery member will be covered and prevented from being accessible from the outside of the medicament delivery device, as shown in FIG. 12. In a preferred example, the lockout structure 33 comprises a track 33a and the housing 1 comprises a track follower 15, e.g., a protrusion, configured to move along the track. A locking ton gue 33b extends from a portion of the track 33a that the track follow can move into that portion after the delivery member cover 3 covers the medicament delivery member again. In one example, the delivery member cover 3 is configured to be moved to cover the medicament delivery member by a biasing member 4. In this example, the locking tongue 33b can be designed to be bent and disengaged from the track follower 15 of the housing when the force that applies to the delivery member cover 3 in the distal direction is greater than a predetermined magnitude. Therefore, the locking tongue 33b can be prevented from being damaged by being squeezed between the delivery member cover 3 and the track follower 15. After the force applies to the delivery member cover 3 in the distal direction is removed or becomes weaker than the force from the biasing member 4, the biasing member will move the delivery member cover 3 back to the position that the delivery member is covered and the locking tongue 3b will engage the track follower 15 again to lock the delivery member cover 3 from being moved in the distal position. This design is configured to prevent a damage to the lockout structure; in particular, when the used medicament delivery device falls. As the damage of the lockout structure might result in that the delivery member cover will not be locked after the medicament delivery device is used, thus might result in an accidental injury to people by a used medicament delivery member. In one example, the locking tongue 33b comprises a lever 33c configured to guide the locking tongue to disengaged from the track follower 15. For example, the locking tongue 33b comprises a distally directed surface 33d configured to engage with the track follower 15 of the housing. In this example, the lever 33c extends beyond the distally directed surface 33d of the locking tongue 33b, as shown in FIG. 11. In this example, the track follower 15 is configured to press the lever 33c in the direction transverse to the longitudinal axis L, and towards the longitudinal axis when the track follower 15 is engaged with the distally directed surface 33d of the locking tongue 33b. Therefore, when the force that applies to the delivery member cover 3 in the distal direction is greater than the predetermined magnitude and the distally directed surface 33d is engaged with the track follower 15, the lever will be pressed further towards the longitudinal axis L and thus pulls the locking tongue towards the longitudinal axis L to disengage from the track follower 15.

The cap 2; 2′ is releasably attached to the tubular section 30; 30′ of the delivery member cover 3; 3′. The cap 2; 2′ operably connects to the sterile seal of the medicament container of the cassette unit, so that when the cap 2; 2′ is removed the sterile seal of the medicament container of the cassette unit is broken. The cap 2; 2′ comprises a user-accessible section 20; 20′, so that the user can grip on the user-accessible section 20; 20′ to remove the cap 2; 2′ from the delivery member cover 3; 3′. In a preferred example, the cap 2 comprises a gripping arm 22 extending towards the distal end of the tubular housing 1. The gripping arm 22 comprises a proximally directed surface configured to engage with a delivery member sheath of a medicament container.

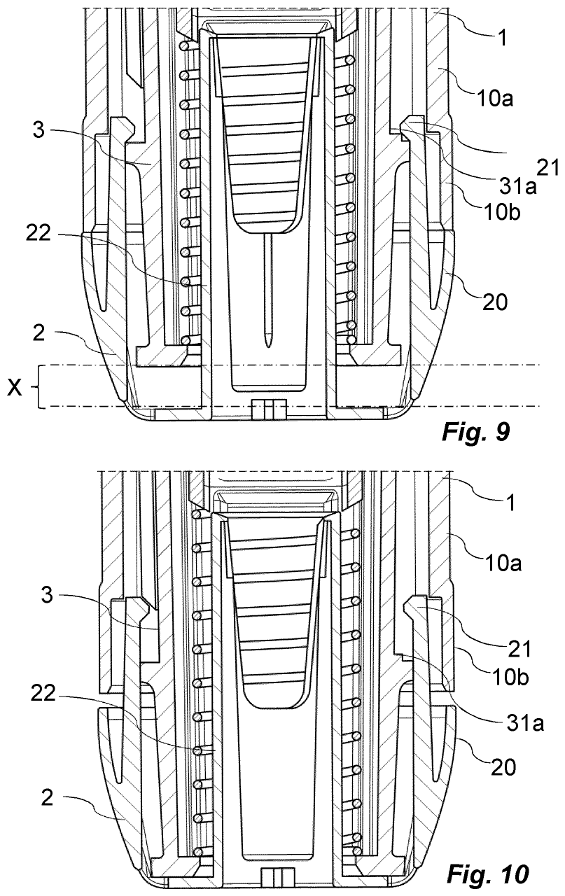

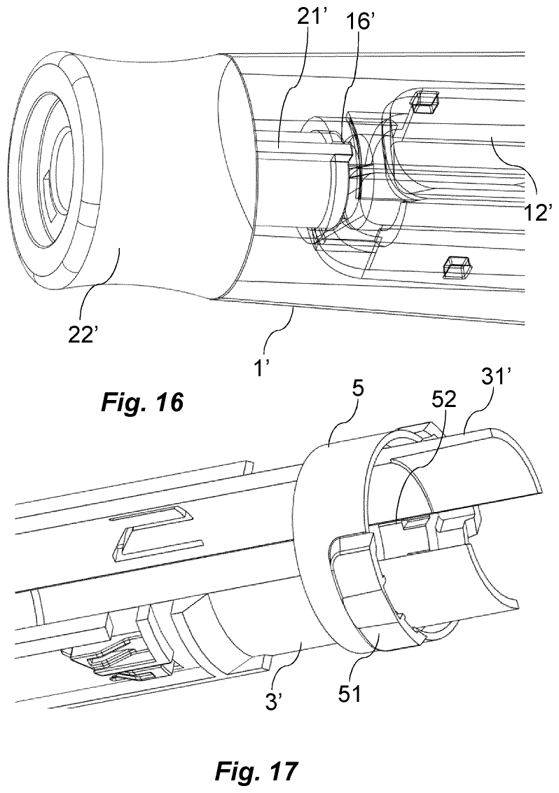

The delivery member cover 3; 3′ is configured to lock the cap 2; 2′ to the housing 1; 1′. The delivery member cover 3; 3′ is movable between a first position where the cap 2; 2′ is locked to the housing 1; 1′ by the delivery member cover 3; 3′ such that the user cannot remove the cap 2; 2′ from the housing 1; l′ and a second position where the cap 2; 2′ is no longer locked to the housing 1; 1′ by the delivery member cover 3; 3′. The delivery member cover 3; 3′ comprises a the distally directed surface 31a; 31a′ and the cap 2; 2′ comprises a proximally directed surface 21a; 21a′. When the delivery member cover 3; 3′ is in the first position, the distally directed surface 31a; 31a′ of the delivery member cover 3; 3′ is adjacent to the proximally directed surface 21a; 21a′ of the cap 2; 2′, as shown in FIG. 9 and FIG. 16. When the delivery member cover 3; 3′ is in the second position, the distally directed surface 31a; 31a′ of the delivery member cover 3; 3′ is away from the proximally directed surface 21a; 21a′ of the cap 2; 2′ such that the cap 2; 2′ can be removed from the delivery member cover 3; 3′ by being moved in the proximal direction relative to the delivery member cover 3; 3′, as shown in FIG. 10.

In one example, the delivery member cover 3 is movable in the direction of the longitudinal axis L relative to the housing 1 between a distal position (shown in FIG. 9) and a proximal position (shown in FIG. 10). The safety mechanism is provided that the cap 2 cannot be detached from the tubular section 30 of the delivery member cover 3 when the delivery member cover 3 is in the distal position such that the delivery member cover is in the first position, and can be detached from the tubular section 30 of the delivery member cover 3 when the delivery member cover moves a distance X into the in the proximal direction relative to the housing 1 into the proximal position such that the delivery member cover is moved to the second position.

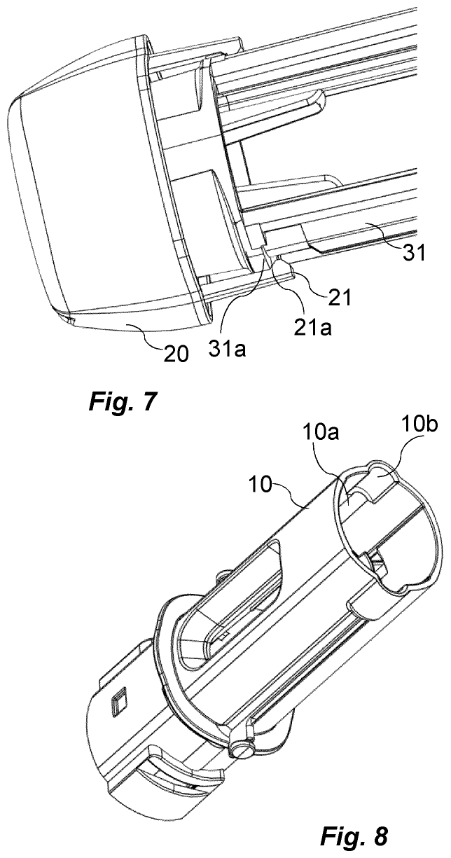

The delivery member cover comprises the distally directed surface 31a and the cap comprises the proximally directed surface 21a adjacent to the distally directed surface 31a of the delivery member cover 3. At least one of the distally directed surface and the proximally directed surface is a chamfered surface bevelled relative to the longitudinal axis L. In the preferred example as shown in FIG. 7, the proximally directed surface 21a is a chamfered surface bevelled relative to the longitudinal axis L. Preferably, the bevelled angle can be 30-70 degrees, more preferred angle can be 40-60 degrees. In a preferred example, the proximally directed surface 21a is formed on a protrusion that extends from an inner surface of a flexible wall 21 of the cap 2 in the direction transverse to the longitudinal axis L. Alternatively, the distally directed surface can be formed on a protrusion that extends from a flexible wall of the delivery member cover. In a preferred example, the flexible wall 21 of the cap 2 is a flexible arm 21 extending from the user-accessible section in the direction of the longitudinal axis L towards the distal end of the housing 1. In a preferred example, the protrusion is arranged at the distal end of the flexible arm 21.

In one example where the cap 2 comprises the gripping arm 22, the gripping arm 22 of the cap 2 is arranged within the delivery member cover 3, and the flexible arm 21 of the cap 2 is arranged between the tubular housing 1 and the delivery member cover 3, as shown in FIGS. 9-10.

The housing 1 comprises a support surface 10a defined by an inner surface of a wall of the housing body 10. The support surface 10a is closer to the proximal end of the housing 1 than the distal end of the housing 1. The support surface 10a is arranged next to a proximal inner surface 10b of the housing body 10. In a preferred example, the support surface 10a is closer to the distal end of the housing 1 than the proximal inner surface 10b. In a preferred example, the support surface 10a is closer to the longitudinal axis than the proximal inner surface 10b of the housing body 10 as shown in FIGS. 8-10. In a preferred example, the housing 1 comprises two support surfaces 10a and two proximal inner surfaces 10b. The two support surfaces 10a defines a first transverse distance. The first transverse distance is shorter than a second transverse distance between the two proximal inner surfaces 10b.

The cap 2 is engaged with the delivery member cover 3 via the engagement between the proximally directed surface 21a and the distally directed surface 31a, so that the cap 2 moves with the delivery member cover 3 from the distal position to the proximal position.

When the delivery member cover 3 is in the distal position, the support surface 10a of the tubular housing 1 is aligned with an outer surface of the flexible wall 21 in the direction transverse to the longitudinal axis L, as shown in FIG. 9. When the delivery member cover 3 is in the proximal position, the support surface 10a of the tubular housing 1 is closer to the distal end of the housing 1 than the flexible wall 21, as shown in FIG. 10. As a result, when the delivery member cover 3 is in the distal position, the flexible wall 21 is blocked from moving in the direction transverse to the longitudinal axis L by the support surface 10a, and when the delivery member cover 3 is in the proximal position, the flexible wall 21 is no longer blocked from moving in the direction transverse to the longitudinal axis L by the support surface 10a. Therefore, if the user pulls the cap 2 in the proximal position when the delivery member cover 3, the bevelled proximally directed surface 21 will divide the axial pulling force to deflect the flexible wall 21, thereby moving the flexible wall in the direction transverse to the longitudinal axis L. When the flexible wall 21 moves in the direction transverse to the longitudinal axis L, the proximally directed surface 21a of the cap 2 is offset from the distally directed surface 31a of the delivery member cover 3, so that the cap 2 can be removed from the delivery member cover 3 as the proximally directed surface 21a of the cap 2 is no longer engaged with the distally directed surface 31a of the delivery member cover 3.

It should be noted that, the proximal inner surface 10b of the housing 1 is dimensioned wider enough to allow the proximally directed surface 21 of the cap 2 can disengage with the distally directed surface 31a of the delivery member cover 3. The exact dimension is of the proximal inner surface 10b of the housing 1 is dependent on the design. Alternatively, instead of the proximal inner surface, an elongated cut-out can be arranged in the proximal end of the housing body. The elongated cut-out is arranged next to the support surface and is close to the proximal end of the housing than the support surface.

In a preferred example, the sub-assembly comprises the biasing member 4 extending along the longitudinal axis L between a proximal end and a distal end. The distal end of the biasing member 4 is adjacent to a proximally directed surface of the tubular housing 1 and the proximal end of the biasing member 4 is adjacent to a distally directed surface of the delivery member cover 3. The biasing member 4 is configured to move the delivery member cover 3 from the distal position to the proximal position.

In a preferred example, the sub-assembly comprises an initial lock mechanism, so that the delivery member cover 3 is locked from moving to the proximal position until the cassette unit that comprises the sub-assembly is attached to the drive unit D. In one example, the initial lock arrangement comprises the flexible section at the distal end of the delivery member cover 3.

In a preferred example, a cut-out 14 is arranged in a wall of the tubular housing 1. The flexible section is movable in the direction transverse to the longitudinal axis L relative to the delivery member cover 3 between an engaged position where the protrusion 32 of the flexible section is positioned within the cut-out 14 of the tubular housing 1 and a released position where the protrusion 32 of the flexible section is positioned out of the cut-out 14 the tubular housing 1. It should be noted that, the cut-out can be replaced by a recess.

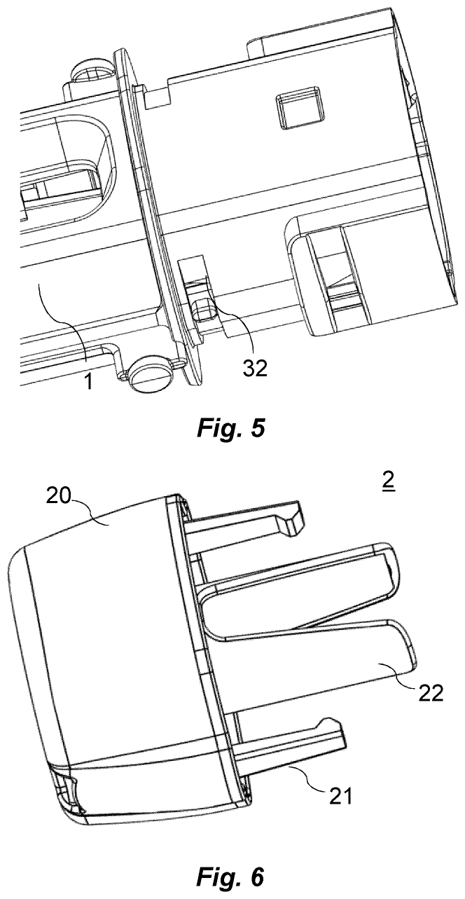

In a preferred example, the flexible section of the delivery member cover 3 is moved from the engaged position to the released position when the protrusion 32 of the flexible section is pressed from the outside of the tubular housing 1. The flexible section of the delivery member cover 3 is configured to be moved from the engaged position to the released position by a section of the drive unit D of the medicament delivery device when the cassette unit with the sub-assembly is attached to the drive unit. Preferably, the flexible section is moved by the drive unit via the protrusion 32. In this example, the protrusion 32 is configured to extend out from the housing 1 through the cut-out 14 of the housing 1, as shown in FIG. 5.

When the cassette unit is inserted into the drive unit D, the section of the drive unit D, e.g. an inner surface of the drive unit moves the protrusion 32 in the direction transverse to the longitudinal axis L, thereby deforming the flexible section. The deformation of the flexible section causes the protrusion 32 to move out from the cut-out 14 of the housing 1, so that the housing 1 is not locked to the delivery member cover 3. As a result, the delivery member cover 3 is moved from the distal position to the proximal position by the biasing member 4.

In another example, the delivery member cover 3′ is configured to be rotated around the longitudinal axis L relative to the housing 1′ from the first position to the second position. In this example, the distally directed surface 31a′ of the delivery member cover 3′ can be provided by a ledge 31′ arranged on an inner surface of the tubular section 30′, as shown in FIGS. 14-15. A groove 31b′ is arranged in the ledge 31′, as shown in FIG. 14. The proximally directed surface 21a′ of the cap 2′ can be arranged at an elongated rib 21′ of the cap 2′. Thus, when the delivery member cover 3′ is in the first position, as shown in FIG. 15, the distally directed surface 31a′ of the delivery member cover 3′ is adjacent to the proximally directed surface 21a′ of the cap 2′; therefore, the cap 2′ cannot be removed from the housing 1′. When the delivery member cover 3′ is rotated to the second position, the distally directed surface 31a′ of the delivery member cover 3′ is no longer adjacent to the proximally directed surface 21a′ of the cap 2′; instead, the the proximally directed surface 21a′ of the cap 2′ is lined up with the groove 31b′; therefore, the cap 2′ can be removed from the housing 1′ by being moved in the proximal direction relative to the housing 1′ and the delivery member cover 3′. In a preferred example, a proximally directed recess 16′ is arranged in a wall of the housing 1′, as shown in FIG. 16. One end of the elongated rib 21′ of the cap 2′ is positioned within the recess 16′ such that the cap 2′ cannot be rotated relative to the housing 1′.

In one example where the sub-assembly comprises the lock ring 5, the lock ring 5 comprises a rotational surface 52 facing in the direction transverse to the longitudinal axis L. The delivery member cover 3′ comprises a counter rotational surface adjacent to the rotational surface 52 of the lock ring 5 such that the delivery member cover is configured to be rotated from the first position to the second position by a rotation of the lock ring 5, as shown in FIG. 17. In a preferred example, the counter rotational surface of the delivery member cover 3′ is a transverse edge of the arm 31′ of the delivery member cover 3′. In one example where the lock ring 5 comprises the fastener 51 configured to be engaged with a counter fastener DO of the drive unit and the fastener 51 and the counter fastener DO formed a bayonet connection, as the lock ring is rotatable relative to the housing 1′, when the user twist the housing 1′ relative to the drive unit for connecting the cassette unit to the drive unit via the bayonet connection, the lock ring 5 rotates the delivery member cover 3′ from the first position to the second position.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are defined by the below clauses.
1. A sub-assembly of a medicament cassette unit, the sub-assembly comprising:
   a tubular housing, a cap, and a delivery member cover;
   wherein the tubular housing extends along a longitudinal axis between a proximal end and a distal end; wherein the tubular housing is configured to receive a medicament container;
   wherein the delivery member cover comprises a tubular section arranged telescopically relative to the proximal end of the tubular housing; wherein the delivery member cover comprises a distally directed surface;
   wherein the cap is releasably attached to the tubular section of the delivery member cover; wherein the cap comprises a proximally directed surface adjacent to the distally directed surface of the delivery member cover;
   wherein at least one of the distally directed surface and the proximally directed surface is a chamfered surface bevelled relative to the longitudinal axis; wherein the distally directed surface is formed on a protrusion that extends from an inner surface of a flexible wall of the delivery member cover in a direction transverse to the longitudinal axis or the proximally directed surface is formed on a protrusion that extends from an inner surface of a flexible wall of the cap in the direction transverse to the longitudinal axis; and wherein the delivery member cover is axially movable relative to the tubular housing from a distal position where a support surface of the tubular housing is aligned with an outer surface of the flexible wall in the direction transverse to the longitudinal axis and a proximal position where the support surface of the tubular housing is closer to the distal end of the housing than the flexible wall.
2. The sub-assembly according to clause 1, wherein the flexible wall is defined by a flexible arm extending in the direction of the longitudinal axis.
3. The sub-assembly according to clause 2, wherein the cap comprises the flexible arm extending towards the distal end of the housing.
4. The sub-assembly according to any one of the preceding clauses, wherein the distally directed surface of the delivery member cover extends from an outer surface of the tubular section of the delivery member cover.
5. The sub-assembly according to any one of the preceding clauses, the sub-assembly comprising a biasing member extending along the longitudinal axis between a proximal end and a distal end; and wherein the distal end of the biasing member is adjacent to a proximally directed surface of the tubular housing and the proximal end of the biasing member is adjacent to a distally directed surface of the delivery member cover.
6. The sub-assembly according to any one of the preceding clauses, wherein the delivery member cover comprises a ledge extending in the direction transverse to the longitudinal axis; and wherein the distally directed surface of the delivery member cover is defined by the ledge.
7. The sub-assembly according to any one of the preceding clauses, wherein the cap comprises a gripping arm extending towards the distal end of the tubular housing; wherein the gripping arm comprises a proximally directed surface configured to engage with a delivery member sheath of a medicament container.
8. The sub-assembly according to clause 7 when dependent on clause 2 or 3, wherein the gripping arm of the cap is arranged within the delivery member cover; and wherein the flexible wall of the cap is arranged between the tubular housing and the delivery member cover.
9. The sub-assembly according to any one of the preceding clauses, wherein the delivery member cover comprises a flexible section at a distal end of the delivery member cover; and wherein the flexible section comprises a protrusion extending from the delivery member cover towards the tubular housing in the direction transverse to the longitudinal axis.
10. The sub-assembly according to clause 9, wherein a cut-out or recess is arranged in a wall of the tubular housing; and wherein the flexible section is movable in the direction transverse to the longitudinal axis relative to the delivery member cover between an engaged position where the protrusion of the flexible section is positioned within the cut-out or recess of the tubular housing and a released position where the protrusion of the flexible section is positioned out of the cut-out or recess of the tubular housing.
11. The sub-assembly according to clause 10, wherein the flexible section of the delivery member cover is moved from the engaged position where the protrusion of the flexible section is positioned within the cut-out to the released position where the protrusion of the flexible section is positioned out of the cut-out when the protrusion of the flexible section is pressed from the outside of the tubular housing.
12. The sub-assembly according to clause 11, wherein the flexible section of the delivery member cover is configured to be moved from the engaged position to the released position by a section of a drive unit of the medicament delivery device when the cassette unit with the sub-assembly is attached to the drive unit.
13. The sub-assembly according to clause 12, wherein the flexible section is moved by the drive unit via the protrusion.
14. The sub-assembly according to any one of clauses 10-13, wherein the protrusion is configured to extend out from the housing through the cut-out of the housing.
15. A method of operating a medicament delivery device, the method comprising the following steps in the following order:
   providing a medicament cassette unit of the medicament delivery device, wherein the medicament cassette unit comprises the sub-assembly according to clause 14;
   providing a drive unit of the medicament delivery device, wherein the drive unit comprises a section for receiving a part of the medicament cassette unit;
   inserting the part of the medicament cassette unit into the section of the drive unit, thereby engaging the flexible section of the delivery member cover of the medicament cassette unit with an inner surface of the drive unit;
   moving the delivery member cover from the distal position to the proximal position; and pulling the cap in the proximal direction relative to the tubular housing, thereby removing the cap from the tubular housing.
16. A sub-assembly of a medicament cassette unit, the sub-assembly comprising:
   a tubular housing, a cap, and a delivery member cover;
   wherein the tubular housing extends along a longitudinal axis between a proximal end and a distal end; wherein the tubular housing is configured to receive a medicament container;
   wherein the delivery member cover comprises a tubular section arranged telescopically relative to the proximal end of the tubular housing; wherein the delivery member cover comprises a distally directed surface;
   wherein the cap is releasably attached to the tubular section of the delivery member cover; wherein the cap comprises a proximally directed surface adjacent to the distally directed surface of the delivery member cover;
   and wherein the delivery member cover is movable between a first position where the distally directed surface of the delivery member cover is adjacent to the proximally directed surface of the cap and a second position where the distally directed surface of the delivery member cover is away from the proximally directed surface of the cap such that the cap can be removed from the delivery member cover by being moved in the proximal direction relative to the delivery member cover.
17. The sub-assembly according to clause 16, wherein at least one of the distally directed surface and the proximally directed surface is a chamfered surface bevelled relative to the longitudinal axis; wherein the distally directed surface is formed on a protrusion that extends from an inner surface of a flexible wall of the delivery member cover in a direction transverse to the longitudinal axis or the proximally directed surface is formed on a protrusion that extends from an inner surface of a flexible wall of the cap in the direction transverse to the longitudinal axis; and wherein the delivery member cover is axially movable relative to the tubular housing from a distal position where a support surface of the tubular housing is aligned with an outer surface of the flexible wall in the direction transverse to the longitudinal axis such that the delivery member cover is in the first position and a proximal position where the support surface of the tubular housing is closer to the distal end of the housing than the flexible wall such that the delivery member cover is in the second position.
18. The sub-assembly according to clause 17, wherein the flexible wall is defined by a flexible arm extending in the direction of the longitudinal axis.
19. The sub-assembly according to clause 18, wherein the cap comprises the flexible arm extending towards the distal end of the housing.
20. The sub-assembly according to any one of clauses 16-19, wherein the distally directed surface of the delivery member cover extends from an outer surface of the tubular section of the delivery member cover.
21. The sub-assembly according to any one of clauses 16-20, the sub-assembly comprising a biasing member extending along the longitudinal axis between a proximal end and a distal end; and wherein the distal end of the biasing member is adjacent to a proximally directed surface of the tubular housing and the proximal end of the biasing member is adjacent to a distally directed surface of the delivery member cover.
22. The sub-assembly according to any one of clauses 16-21, wherein the delivery member cover comprises a ledge extending in the direction transverse to the longitudinal axis; and wherein the distally directed surface of the delivery member cover is defined by the ledge.
23. The sub-assembly according to any one of clauses 16-22, wherein the cap comprises a gripping arm extending towards the distal end of the tubular housing; wherein the gripping arm comprises a proximally directed surface configured to engage with a delivery member sheath of a medicament container.
24. The sub-assembly according to clause 23 when dependent on clause 18 or 19, wherein the gripping arm of the cap is arranged within the delivery member cover; and wherein the flexible wall of the cap is arranged between the tubular housing and the delivery member cover.
25. The sub-assembly according to any one of clauses 16-24, wherein the delivery member cover comprises a flexible section at a distal end of the delivery member cover; and wherein the flexible section comprises a protrusion extending from the delivery member cover towards the tubular housing in the direction transverse to the longitudinal axis.
26. The sub-assembly according to clause 25, wherein a cut-out or recess is arranged in a wall of the tubular housing; and wherein the flexible section is movable in the direction transverse to the longitudinal axis relative to the delivery member cover between an engaged position where the protrusion of the flexible section is positioned within the cut-out or recess of the tubular housing and a released position where the protrusion of the flexible section is positioned out of the cut-out or recess of the tubular housing.
27. The sub-assembly according to clause 26, wherein the flexible section of the delivery member cover is moved from the engaged position where the protrusion of the flexible section is positioned within the cut-out to the released position where the protrusion of the flexible section is positioned out of the cut-out when the protrusion of the flexible section is pressed from the outside of the tubular housing.
28. The sub-assembly according to clause 27, wherein the flexible section of the delivery member cover is configured to be moved from the engaged position to the released position by a section of a drive unit of the medicament delivery device when the cassette unit with the sub-assembly is attached to the drive unit.
29. The sub-assembly according to clause 28, wherein the flexible section is moved by the drive unit via the protrusion.
30. The sub-assembly according to any one of clauses 26-29, wherein the protrusion is configured to extend out from the housing through the cut-out of the housing.
31. The sub-assembly according to clause 16, wherein the sub-assembly comprises a lock ring attached to the housing; wherein the lock ring is axially immovable and rotatable around the longitudinal axis relative to the housing; wherein the lock ring comprises a rotational surface facing in the direction transverse to the longitudinal axis; wherein the delivery member cover comprises a counter rotational surface adjacent to the rotational surface of the lock ring such that the delivery member cover is configured to be rotated from the first position to the second position by a rotation of the lock ring.
32. The sub-assembly according to clause 31, wherein the lock ring comprises a fastener configured to be engaged with a counter fastener of the drive unit; and wherein the fastener and the counter fastener formed a bayonet connection.

1-15. (canceled)

16. A sub-assembly of a medicament cassette unit, the sub-assembly comprising:
a tubular housing;
a cap; and
a delivery member cover,
wherein the tubular housing extends along a longitudinal axis between a proximal end and a distal end, wherein the tubular housing is configured to receive a medicament container;
wherein the delivery member cover comprises a tubular section arranged telescopically relative to the proximal end of the tubular housing, wherein the delivery member cover comprises a distally directed surface,
wherein the cap is releasably attached to the tubular section of the delivery member cover, wherein the cap comprises a proximally directed surface adjacent to the distally directed surface of the delivery member cover, and
wherein the delivery member cover is movable between a first position where the distally directed surface of the delivery member cover is adjacent to the proximally directed surface of the cap and a second position where the distally directed surface of the delivery member cover is away from the proximally directed surface of the cap such that the cap can be removed from the delivery member cover by being moved in the proximal direction relative to the delivery member cover.

17. The sub-assembly according to claim 16, wherein at least one of the distally directed surface and the proximally directed surface is a chamfered surface bevelled relative to the longitudinal axis, wherein the distally directed surface is formed on a protrusion that extends from an inner surface of a flexible wall of the delivery member cover in a direction transverse to the longitudinal axis or the proximally directed surface is formed on a protrusion that extends from an inner surface of a flexible wall of the cap in the direction transverse to the longitudinal axis, and wherein the delivery member cover is axially movable relative to the tubular housing from a distal position where a support surface of the tubular housing is aligned with an outer surface of the flexible wall in the direction transverse to the longitudinal axis such that the delivery member cover is in the first position and a proximal position where the support surface of the tubular housing is closer to the distal end of the housing than the flexible wall such that the delivery member cover is in the second position.

18. The sub-assembly according to claim 17, wherein the flexible wall is defined by a flexible arm extending in the direction of the longitudinal axis.

19. The sub-assembly according to claim 18, wherein the cap comprises the flexible arm extending towards the distal end of the housing.

20. The sub-assembly according to claim 16, wherein the distally directed surface of the delivery member cover extends from an outer surface of the tubular section of the delivery member cover.

21. The sub-assembly according to claim 16, the sub-assembly comprising a biasing member extending along the longitudinal axis between a proximal end and a distal end, and wherein the distal end of the biasing member is adjacent to a proximally directed surface of the tubular housing and the proximal end of the biasing member is adjacent to a distally directed surface of the delivery member cover.

22. The sub-assembly according to claim 16, wherein the delivery member cover comprises a ledge extending in the direction transverse to the longitudinal axis, and wherein the distally directed surface of the delivery member cover is defined by the ledge.

23. The sub-assembly according to claim 16, wherein the delivery member cover comprises a flexible section at a distal end of the delivery member cover, and wherein the flexible section comprises a protrusion extending from the delivery member cover towards the tubular housing in the direction transverse to the longitudinal axis.

24. The sub-assembly according to claim 23, wherein a cut-out or recess is arranged in a wall of the tubular housing, and wherein the flexible section is movable in the direction transverse to the longitudinal axis relative to the delivery member cover between an engaged position where the protrusion of the flexible section is positioned within the cut-out or recess of the tubular housing and a released position where the protrusion of the flexible section is positioned out of the cut-out or recess of the tubular housing.

25. The sub-assembly according to claim 24, wherein the flexible section of the delivery member cover is moved from the engaged position where the protrusion of the flexible section is positioned within the cut-out to the released position where the protrusion of the flexible section is positioned out of the cut-out when the protrusion of the flexible section is pressed from the outside of the tubular housing.

26. The sub-assembly according to claim 25, wherein the flexible section of the delivery member cover is configured to be moved from the engaged position to the released position by a section of a drive unit of the medicament delivery device when the cassette unit with the sub-assembly is attached to the drive unit.

27. The sub-assembly according to claim 26, wherein the flexible section is moved by the drive unit via the protrusion.

28. The sub-assembly according to claim 24, wherein the protrusion is configured to extend out from the housing through the cut-out of the housing.

29. The sub-assembly according to claim 16, wherein the sub-assembly comprises a lock ring attached to the housing, wherein the lock ring is axially immovable and rotatable around the longitudinal axis relative to the housing, wherein the lock ring comprises a rotational surface facing in the direction transverse to the longitudinal axis, wherein the delivery member cover comprises a counter rotational surface adjacent to the rotational surface of the lock ring such that the delivery member cover is configured to be rotated from the first position to the second position by a rotation of the lock ring.

30. The sub-assembly according to claim 29, wherein the lock ring comprises a fastener configured to be engaged with a counter fastener of the drive unit, and wherein the fastener and the counter fastener formed a bayonet connection.
